(19) United States
(12) Patent Application Publication
(10) Pub. No.: US 2008/0248542 A1
(43) Pub. Date: Oct. 9, 2008

(54) Clostridium difficile culture and toxin production methods

(76) Inventors: Arnold L. Demain, Madison, NJ (US); Aiqi Fang, Somerville, MA (US)

Correspondence Address: Pearl Cohen Zedek Latzer, LLP, 1500 Broadway, 12th Floor, New York, NY, 10036, US

(21) Appl. No.: 12/068,544

(22) Filed: Feb. 7, 2008

Related U.S. Application Data
(62) Division of application No. 10/743,569, filed on Dec. 22, 2003, now abandoned.
(60) Provisional application No. 60/436,378, filed on Dec. 23, 2002.

(51) Int. Cl. C12P 1/00; C12N 1/20

(52) U.S. Cl. 435/170; 435/252.7

(57) ABSTRACT

The invention provides methods and compositions for use in culturing Clostridium difficile and producing Clostridium difficile toxins.

### CROSS REFERENCE TO RELATED APPLICATIONS

This application is a divisional of pending U.S. patent application Ser. No. 10/743,569 filed on Dec. 22, 2003, which claims priority from U.S. Provisional Patent Application No. 60/436,378, filed Dec. 23, 2002, now expired, both which are incorporated herein by reference in their entirety.

### FIELD OF INVENTION

This invention relates to methods for culturing Clostridium difficile and producing Clostridium difficile toxins.

### BACKGROUND OF THE INVENTION

Clostridium difficile is a gram-positive, spore-forming, toxigenic bacterium that causes antibiotic-associated diarrhea, which can progress into severe and sometimes fatal colitis. These conditions can occur when the normal intestinal flora is disrupted by, e.g., antibiotic or anti-neoplastic therapy. Such disruption enables C. difficile to become established in the colon, where it produces the causative agents of these conditions: two high molecular weight toxins, Toxin A and Toxin B. Both of these polypeptides are cytotoxins, but Toxin B is greater than 1,000-fold more potent than Toxin A. Toxin A is also an enterotoxin, as it causes accumulation of fluid in ligated animal intestinal loops.

C. difficile Toxins A and B are encoded by two separate but closely linked genes that together form part of a 19.6 kilobase region known as the “toxigenic element” or the “pathogenicity locus.” The Toxin A and B genes and proteins are highly homologous, as it is likely that the genes evolved by duplication. Toxins A and B are produced simultaneously in C. difficile strain VPI 10463 (ATCC 43255), and the ratio of the produced toxins is usually 3:1, respectively (Karlsson et al., Microbiology 145:1683-1693, 1999). The toxins begin to be formed during the exponential growth phase, and they are usually released from the bacteria between 36 and 72 hours of culture. Toxins present within the bacteria can be released earlier by sonication or by use of a French pressure cell.

Media for the growth of C. difficile typically contain animal and dairy by-products as sources of proteins, amino acids, and other nutrients required for growth (see, e.g., Holbrook et al., J. Appl. Bacteriol. 42:259-273, 1977). Manufacturers of such media have used complex ingredients, such as casein digests and meat extracts, to maximize toxin production.

### SUMMARY OF THE INVENTION

In one embodiment, the invention provides methods of culturing C. difficile, which involve growing the C. difficile in media that are substantially free of animal-derived products (e.g., media that lack animal-derived products). These media can include one or more compounds derived from a vegetable (e.g., a soybean), such as hydrolyzed soy. These media can also, optionally, include an iron source. The culturing can, optionally, be carried out under anaerobic conditions.

The methods of the invention can be used to grow C. difficile in seed cultures, for example, seed cultures that are started by inoculation from a stock culture that was grown in medium that was substantially free of animal-derived products. The methods can also be used to grow C. difficile in fermentation cultures, which can have been inoculated from seed cultures (e.g., first or second seed cultures) that were grown in medium that was substantially free of animal-derived products. These methods can further include isolating C. difficile toxins (i.e., Toxin A and/or Toxin B) from the medium.

The invention also provides methods for obtaining C. difficile toxins. These methods involve (i) culturing C. difficile in a first medium that is substantially free of animal-derived products, under conditions that facilitate growth of C. difficile; (ii) inoculating a second medium that is substantially free of animal-derived products with all or a portion of the first medium after the culturing; (iii) culturing the inoculated second medium under conditions that facilitate growth of C. difficile and toxin production; and (iv) isolating C. difficile toxins from the second medium.

The media used in these methods can include one or more compounds that are derived from a vegetable (e.g., a soybean), such as hydrolyzed soy. Any or all of the culturing steps of these methods can, optionally, be carried out under anaerobic conditions. Moreover, culturing in the first medium can be started by inoculation with a previous C. difficile culture (e.g., a stock culture or a previous seed culture) that was cultured in medium that was substantially free of animal-derived products.

Also included in the invention are compositions that include a culture medium that is substantially free of animal products and also contain Clostridium difficile. These compositions can also include one or more compounds that are derived from a vegetable (e.g., a soybean), such as hydrolyzed soy. Optionally, these compositions can also include an iron source.

The invention provides several advantages. For example, use of animal product-free media provides an important safeguard against the possibility of contamination of medical products (e.g., vaccines) that are derived from the cultured bacteria with undesirable material. Such contaminants include, for example, the causative agent of Bovine Spongoform Encephalopathy (i.e., mad cow disease or BSE), antigenic peptides that stimulate undesired immune reactions in immunized subjects (e.g., anaphylactic reactions), and viruses. The invention is also advantageous because it facilitates high efficiency bacterial growth and toxin production. Other features and advantages of the invention will be apparent from the following detailed description and the claims.

Other features and advantages of the present invention will become apparent from the following detailed description examples. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

The invention provides methods and compositions for use in growing Clostridium difficile and producing the C. difficile toxins, toxins A and B. These toxins can be used, for example, in vaccination methods or in the preparation of toxoids, which can in turn be used in vaccines. As is discussed further below, the methods and compositions of the invention employ culture media that contain significantly reduced levels of animal products, such as meat or dairy by-products, if any. The invention is based on the present inventors' discovery that animal-based products, which traditionally have been used in media for culturing C. difficile, are not required to achieve efficient culture of these bacteria. As is described in further detail below, the inventors found that vegetable-based products can replace animal-based products in these media, leading to high levels of bacterial growth and toxin production. As is noted above, replacing the animal components of culture media with vegetable-based products reduces the potential for contamination of medical products made from the bacteria (e.g., toxins, toxoids, and cell preparations) with undesirable molecules, such as certain proteins and viruses that may exist in animals.

The vegetable-based products in the media used in the present invention can be, for example, soy-based products. The soy-based products can be, optionally, hydrolyzed and, preferably, are soluble in water. However, insoluble soy products can also be used in the methods of the invention. Common animal products that can be substituted by soy products include beef heart infusion (BHI), peptones (e.g., tryptones), and dairy by-products, such as animal milk, or casein or its hydrosylates. Examples of soy products that can be used in the invention, and their sources, include:
I. Tekniscience: Soy Peptone A1, Soy Peptone A2, Soy Peptone A3, Plant Peptone E1, Plant Peptone ET1, and Wheat Peptone E1;
II. Quest: HY-Soy, HY-Soy T, AMI-Soy, NZ-Soy, NZ-Soy BLA, and NZ-Soy BL7;
   III. DMV: SE50M, SE70M, SE50MK, WGE80BT, WGE80M, CNE50M, and SE70BT;
   IV. Marcor: Soy Peptone Type AB, Soy Peptone Type AC, Soy Peptone Type SL, Soy Peptone Type II, and Soy Peptone Type F;
   V. Oxoid: Vegetable Peptone and Vegetable Peptone No. 1;
   VI. Gibco: Soy Peptone; and
   VII. Difco: Bacsoytone.

Media containing vegetable (e.g., soy) products for the growth of C. difficile can be similar to commonly used growth media containing animal derived products (e.g., TY or TYM media; see below), except that all or substantially all of the animal-derived products are replaced with the vegetable-derived products. In addition, ingredients in TY, TYM, and similar media that are not essential for growth of C. difficile in media containing soy-based products can also be included in the media nonetheless, to enhance growth and toxin production.

In one embodiment, growth of C. difficile according to the methods of the invention proceeds in at least two phases: seed growth and fermentation. A relatively small seed culture is first grown by inoculation from a stock culture, e.g., a working cell bank, and this seed is used either to inoculate a second seed culture or to inoculate a relatively large fermentation culture. As is well understood in the art, the number of seed cultures used depends, for example, on the size and volume of the fermentation step. As is described further below, the fermentation media used in the methods of the invention lacks animal products, as described herein. Preferably, the seed and stock cultures employ media that lack such products as well, although this is not absolutely required.

In another embodiment, the culturing stages of the methods of the invention (both seed and fermentation) are carried out under anaerobic conditions, although aerobic conditions for either of these phases may be used as well. Approaches to anaerobic culture of bacteria, such as C. difficile, are well known in the art and can employ, for example, nitrogen gas or a mixture of nitrogen and hydrogen gases. The gas can either be bubbled through the medium during fermentation or passed through the area above the liquid in a culture chamber (i.e., the chamber headspace). Preferably, the nitrogen gas or nitrogen/hydrogen gas mixture is passed through the headspace in a continuous manner.

The seed growth phase (or phases) are generally carried out to scale-up the quantity of the microorganism from a stored culture, so that it can be used as an inoculant for the fermentation phase. The seed growth phase can also be carried out to allow relatively dormant microbes in stored cultures to become rejuvenated and to grow into actively growing cultures. Further, the volume and quantity of viable microorganisms used to inoculate the fermentation culture can be controlled more accurately if taken from an actively growing culture (i.e., a seed culture), rather than if taken from a stored culture. In addition, as is noted above, more than one (e.g., two or three) seed growth phases can be used to scale-up the quantity of C. difficile for inoculation of the fermentation medium. Alternatively, growth of C. difficile in the fermentation phase can proceed directly from the stored culture by direct inoculation, if desired.

To start the fermentation phase, a portion or all of a seed culture containing C. difficile is used to inoculate fermentation medium. Fermentation is used to produce the maximum amount of the bacterium in a large-scale anaerobic environment (Ljungdahl et al., “Manual of Industrial Microbiology and Biotechnology,” 1986, ed., Demain and Solomon, American Society for Microbiology, Washington, D.C., p. 84).

C. difficile toxins can be isolated and purified from fermentation cultures using well known protein purification methods. (See, e.g., Coligan et al., “Current Protocols in Protein Science,” Wiley & Sons; Ozutsumi et al., Appl. Environ. Microbiol. 49:939-943, 1985; and Kim et al., Infection and Immunity 55:2984-2992, 1987; which are incorporated herein by reference.) The purified toxins can then, for example, be inactivated by formaldehyde treatment, so that they can be used, e.g., in immunization methods (see, e.g., Libby et al., Infection and Immunity 36:822-829, 1982).

Additional details concerning the methods of the invention are provided as follows.

### Seed Culture

As is discussed above, cultures of C. difficile can be grown in one or more seed cultures to produce a sufficient quantity of active cultures for the inoculation of fermentation medium. Also as is discussed above, the number of steps involving growth in a seed medium (e.g., inoculation of a second seed medium by a first seed culture) can vary, depending on the scale of the production in the fermentation phase. An example of an animal-based seed medium that has been used to culture C. difficile is TYM, which includes tryptone peptone, yeast extract, mannitol, and glycerol (see below). This medium can be adapted for use in the present invention by replacing the tryptone peptone with a vegetable-derived product, such as a soy-based product. For example, a hydrolyzed soy product, which may be soluble in water, can be used. Any source of such soy-based products may be used in the present invention including, for example, those listed above (e.g., NZ Soy BLA or Soy Peptone A3).

Concentrations of the soy product in the seed medium can range, for example, between 5 and 200 g/L, e.g., 20-150 g/L, 25-100 g/L, or 50-75 g/L. Concentrations of a carbon source (e.g., glucose, mannitol, or glycerol) in this medium can range, for example, between 0.1 g/L and 20.0 g/L, e.g., 0.5-10.0 g/L or 1-5 g/L. Any combination of carbon sources can be used in the medium. For example, as with TYM medium, mannitol and glycerol can both be included.

To enhance toxin A production, an iron compound, such as, for example, reduced iron powder (e.g., 0.1-5.0 g/L, 0.25-3.0 g/L, or 0.5-1.5 g/L), FeSO₄.7H₂O (e.g., 1-100 mg/L or 40-60 mg/L), or ferrous gluconate (e.g., 50-400 mg/L, 150-300 mg/L, or 200-250 mg/L), can be included in the seed culture media. Additional examples of iron sources that can be used in the invention include non-reduced iron powder (J. T. Baker and Sigma-Aldrich), iron wire (e.g., Puratronic, Alfo Aesoar A. Johnson Matthey Co., and Sigma-Aldrich), iron foil, ferric citrate, and ferrous ammonium sulfate. When iron powder is used, it can be autoclaved together with other ingredients of the fermentation medium. When iron wire is used, it can have a diameter of, for example, between approximately 0.05 mm and 2.0 mm, e.g., a diameter of 0.075 mm (e.g., Puratronic; 99.995% metal basis pure). The preferred pH level of the seed medium prior to growth can range between 6.8 and 8.5, and thus can be, for example, approximately 6.8 or 7.5.

As is noted above, growth of C. difficile in the seed medium may proceed in one or more stages, for example, in two stages. In stage one, a culture of C. difficile is suspended in seed medium and is incubated at a temperature between 30-40° C., preferably 34±1° C., for 24-48 hours in an anaerobic environment. In stage two, a portion or all of the stage one seed medium containing C. difficile is used to inoculate a stage two seed medium for further growth. After inoculation, the stage two medium is incubated at a temperature between 30-40° C., preferably at 34±1° C. or 37±1° C., for approximately 1-4 days, e.g., for 1-2 days, also in an anaerobic environment. Preferably, growth in seed media at any stage does not result in cell lysis before inoculation of fermentation media. Additional growth in a third (or further) stage seed medium can be carried out as well, if desired. An appropriate concentration of seed culture to use to inoculate fermentation media can be determined by those of skill in this art and can range, for example, from 0.1-10%. As specific examples, concentrations of 0.5, 1.0, or 5.0% can be used.

### Fermentation Culture

An example of an animal-based fermentation medium that has been used to culture C. difficile is TY, which includes tryptone peptone, yeast extract, and sodium thioglycolate (see below). This medium can be adapted for use in the present invention by replacing the tryptone peptone with a vegetable-derived product, such as a soy-based product. For example, a hydrolyzed soy product, which preferably is soluble in water, can be used. Any source of such soy-based products can be used including, for example, those listed above (e.g., NZ-Soy BL7, NZ-Soy BIA, NZ Soy, Oxoid Vegetable Peptone No. 1, or WGE80M).

The concentration of soy product in the fermentation medium can range between 5 and 200 g/L, 20-150 g/L, 25-100 g/L, or 50-75 g/L. Optionally, the medium can include an iron source, such as those listed above, in the amounts listed above. The pH of the fermentation medium can range between 7.0 and 8.5. Thus, for example, the pH can be 6.8 or 7.5.

Fermentation can be carried out in an anaerobic chamber at approximately 34±1° C. or 37±1° C. for approximately 4 to 9 days. Growth can be monitored by measuring the optical density (O.D.) of the medium. Fermentation can be stopped after cell lysis has proceeded for at least 48 hours, as determined by growth measurement (optical density). As cells lyse, the O.D. of the medium will decrease.

C. difficile can be cultivated by fermentation with continuous exposure to a 90% nitrogen/10% hydrogen mixture or to 100% nitrogen. Nitrogen gas or a mixture of nitrogen and hydrogen gas may also be bubbled through the medium during fermentation. In addition, agitation (approximately 100 rpm) of the culture during fermentation can be used. General methods of fermentation for C. difficile are well known to those skilled in the art, and can be used in the invention.

### Stock Culture

As is noted above, the media in which stock cultures (i.e., working cell bank cultures) for inoculating seed cultures is present can include, optionally, vegetable products in place of animal products as described herein. For example, the media can include a soy product in place of tryptone peptone in TYM medium (see below). The soy product can be any of those listed above, e.g., Soy Peptone A3 or NZ-Soy BL4.

According to another alternative, cultures of C. difficile used for long-term storage and for inoculation of seed media can be grown and lyophilized in soy-milk prior to storage at 4° C. However, to maintain media that are substantially free of animal by-products throughout the production of C. difficile toxins, it is preferred that the initial culture of C. difficile be preserved in soy milk, and not animal milk. The stored culture, which can be lyophilized, is thus produced by growth in media containing proteins derived from soy and lacking animal by-products. Growth of C. difficile in fermentation medium can proceed by inoculation directly from such a stored, lyophilized culture, or through seed cultures, as is discussed above.

The following examples are presented in order to more fully illustrate the preferred embodiments of the invention. They should in no way be construed, however, as limiting the broad scope of the invention.

### EXAMPLES

### Materials and Methods

### Seed

i) Tryptone-Yeast extract-Mannitol Medium (TYM, g/L)

| | |
|---|---|
| Tryptone peptone | 24 |
| Yeast Extract | 12 |
| Mannitol | 10 |
| Glycerol | 1 |
| pH | 6.8 |

ii) Preparation of Seed Medium:

Tryptone peptone (Difco) 2.4 g, yeast extract 1.2 g, mannitol 1 g and glycerol 0.1 g were added into 100 ml d.d. water. pH was adjusted to 6.8 with 5N NaOH.

10 ml seed medium was dispensed into each seed tube (16×150 mm) and 40 ml seed medium into each 2510-DeLong Bellco Culture Flask (125 ml).

Autoclaving was done at 121° C. for 30 minutes, and the vessels were then immediately moved to a COY Anaerobic Chamber (Coy Laboratory Products Inc., Grass lake, Mich.) filled with 10% carbon dioxide (CO₂) plus 10% hydrogen (H₂) and 80% nitrogen (N₂).

iii) Seed Culture:

First stage seed culture: A vial of working cell bank (WCB) culture (1 ml) was transferred into a 16×150 mm test tube containing 10 ml seed medium (TYM) and incubated at 35±1° C. for 24 hours.

Second stage seed culture: 1 ml first stage seed culture was added as an inoculum to a 125 ml DeLong Bellco Culture Flask containing 40 ml seed medium (TYM). The flasks were incubated at 37±1° C. for 24 hours.

### Fermentation

i) Fermentation Media

A. Tryptone-Yeast extract-
Mannitol Medium (TYM, g/L)

| | |
|---|---|
| Tryptone peptone (Difco) | 24 |
| Yeast extract | 12 |
| Mannitol | 10 |
| Glycerol | 1 |
| pH | 6.8 |

B. TY medium (g/L):

| | |
|---|---|
| Tryptone peptone (Difco) | 30 |
| Yeast extract | 20 |
| Sodium thioglycolate | 1 |
| pH | 6.8 |

C. TYG medium (g/L):

| | |
|---|---|
| Tryptone peptone (Difco) | 30 |
| Yeast extract | 20 |
| Glucose | 10 |
| Sodium thioglycolate | 1 |
| pH | 6.8 |

D. Tryptone-Yeast extract-
Mannitol Medium (TYM-2, g/L)

| | |
|---|---|
| Tryptone peptone (Difco) | 12 |
| Yeast extract | 24 |
| Mannitol | 10 |
| Glycerol | 5 |
| pH | 8.0 |
| | (adjust pH with KOH) |

ii) Non-Animal Peptones:
I. Tekniscience: Soy Peptone A1, Soy Peptone A2, Soy Peptone A3, Plant Peptone E1, Plant Peptone ET1, and Wheat Peptone E1;
II. Quest: HY-Soy, HY-Soy T, AMI-Soy, NZ-Soy, NZ-Soy BLA, and NZ-Soy BL7;
III. DMV: SE50M, SE70M, SE50MK, WGE80BT, WGE80M, CNE50M, and SE70BT;
IV. Marcor: Soy Peptone Type AB, Soy Peptone Type AC, Soy Peptone Type SL, Soy Peptone Type II, and Soy Peptone Type F; and
V. Oxoid: Vegetable Peptone and Vegetable Peptone No. 1.

iii) Preparation of Fermentation Media:

At first, 3 g peptones were individually placed into 150 ml bottles. The fermentation medium was prepared without the peptones and 100 ml medium was added to each bottle, dissolved with a magnetic stirring bar, and then the pH was adjusted to 6.8 with 3N HCl or SN NaOH. Autoclaving was done at 121° C. for 30 minutes, then the bottles were immediately moved to a COY Anaerobic Chamber filled with 10% carbon dioxide (CO₂) plus 10% hydrogen (H₂) and 80% nitrogen (N₂). Eight ml medium was added to each 16×100 mm test tube.

iv) Cultivation

40 μl seed culture (step 2) was used as an inoculum (0.5%) for each 8 ml of production medium in 16×100 mm test tubes. Three tubes were used for each variable. The tubes were incubated in the anaerobic chamber at 37±1° C. for 5 days. Growth (OD) was measured before mixing and after mixing (excepting the case of insoluble peptones) with a Turner Spectrophotometer (Model 330) at 540 nm after 24 hours after inoculation. One uninoculated tube was used as a blank to zero in the spectrophotometer. The cultivation was usually stopped on the 3^{rd} and the 5^{th} day.

### Toxin Production was Measured by the ELISA Method

A Fusion Universal Microplate Analyzer (Packard, Meriden, Conn.) was used for reading of the ELISA plates using filters 405 nm and 490 nm.

Microorganism: Clostridium difficile VP110463

### Example 1

### Comparison of Different Complex Fermentation Media for Toxin A Production

TABLE I
Cell growth and Toxin A production in the different fermentation media:
TYM medium (control), TY medium, and TY medium + 10 g/L glocose.

| | |
|---|---|
| | Toxin A |
| Cell growth | production (ng/ml) |

| Media | (OD_{540 nm} at 24 hours) | 5 days | 7 days |
|---|---|---|---|
| TYM | 0.97 | 27 | 47 |
| TY | 0.95 | 2500 | 2420 |
| TYG | 1.05 | 26 | 67 |

Table 1 shows that Toxin A production was best in TY medium, which contains 1 g/L sodium thioglycolate. Glucose slightly increased cell growth, but markedly inhibited Toxin A production.

TABLE II
Cell growth and Toxin A production
in TYM-2 fermentation medium.

| | |
|---|---|
| | Toxin A |
| Cell growth | production (ng/ml) |

| Media | (OD_{540 nm} at 24 hours) | 3 days | 5 days |
|---|---|---|---|
| TYM-2 | 1.20 | <160 | <160 |
| TY | 0.79 | 6818 | 8416 |

Table 2 shows that cell growth was increased, but Toxin A production was markedly inhibited, in TYM-2 fermentation medium, which is similar to TYM, but contains higher levels of glycerol and yeast extract and a lower level of Tryptone.

Tables 1 and 2 show that TY is a superior fermentation medium when using Tryptone as a nitrogen source.

### Example 2

### Determination of the Effect of Carbon Sources in the Fermentation Medium on Cell Growth and Toxin A Production

TABLE III
The effect of mannitol and glycerol as carbon sources
on cell growth and Toxin A production in the fermentation
medium with Hy-Soy as replacement for Tryptone.

| | | Toxin A | |
|---|---|---|---|
| Carbon sources | Cell growth | production (ng/ml) | |
| (g/L) | (OD_{540 nm} at 24 hours) | 3 days | 5 days |
| Without carbon | 0.66 | 3700 | 3820 |
| source | | | |
| Glucose (10) | 0.90 | 218 | 244 |
| Mannitol (10) | 0.84 | 158 | 78 |
| Glyceral (1) | 0.68 | 3650 | 3820 |
| Mannitol (10) + | 0.81 | 140 | 78 |
| Glyceral (1) | | | |

Table 3 shows that glucose and mannitol markedly inhibited Toxin A production, but glycerol.

### Example 3

### Determination of Whether Non-Animal Peptone Products can Replace Tryptone in Fermentation Medium for Cell Growth and Toxin a Production

TABLE IV
Comparison of different non-animal peptones as replacements
for Tryptone in TYM fermentation medium.

| | | Toxin A | |
|---|---|---|---|
| | Cell growth | production (ng/ml) | |
| Peptone | (OD_{540 nm} at 24 hours) | 5 days | 7 days |
| Tryptone | 0.97 | 27 | 47 |
| Hy-Soy | 0.76 | 66 | 80 |
| SE50MK | 0.59 | <6 | <6 |
| Soy peptone A1 | 0.78 | <6 | <6 |
| Soy peptone A2 | 0.86 | 25 | 24 |
| Soy peptone A3 | 0.91 | 29 | 28 |
| Plant peptone E1 | 0.82 | 64 | 34 |
| Plant peptone ET1* | — | <6 | <6 |
| Wheat peptone E1 | 0.72 | <6 | <6 |

Table 4 shows that Hy-Soy was the best choice of 8 different non-animal peptones in TYM fermentation medium as a Tryptone replacement for Toxin A production. However, TYM is not the medium of choice for fermentation. Thus, we continued our examination of peptones in TY medium, less Tryptone.

TABLE V
Comparison of different non-animal peptones as replacements
for Tryptone in TY fermentation medium.

| | | Toxin A | |
|---|---|---|---|
| | Cell growth | production (ng/ml) | |
| Peptone | (OD_{540 nm} at 24 hours) | 3 days | 5 days |
| Tryptone | 0.88 | 1700 | 4250 |
| Hy-Soy | 0.66 | 3700 | 3820 |
| Plant peptone E1* | — | 2700 | 4000 |
| Vegetable Peptone | 1.07 | 218 | 288 |
| Vegetable Peptone No. 1 | 0.75 | 2580 | 10000 |
| Soy Peptone Type II | 0.64 | 4200 | 4250 |
| Soy Peptone Type AC | 0.79 | 3650 | 3475 |
| Soy Peptone Type AB | 0.77 | 4000 | 3110 |
| Soy Peptone Type SL | 0.48 | 2860 | 4020 |
| Soy Peptone Type F* | — | 4020 | 4250 |

*The peptone is insoluble.

Table 5 shows that TY is a much better fermentation medium than TYM (compare to titers in Table 4) and that Vegetable Peptone No. 1 was the best choice of 9 different non-animal peptones tested to replace Tryptone in fermentation medium for Toxin A production. It was better than Hy-Soy, which was the best in the poor TYM medium (Table 4).

TABLE VI
Comparison of different non-animal peptones as replacements
for Tryptone in TY fermentation medium.

| | | Toxin A | |
|---|---|---|---|
| | Cell growth | production (ng/ml) | |
| Peptone | (OD_{340 nm} at 24 hours) | 3 days | 5 days |
| Tryptone | 0.86 | 2480 | 3900 |
| NZ-Soy | 0.96 | 7000 | 4420 |
| NZ-Soy BL4 | 0.83 | 9000 | 4500 |
| NZ-Soy BL7* | — | 10000 | 9875 |
| AMI-Soy* | — | 2950 | 3500 |
| HY-Soy T* | — | 2150 | 4100 |
| SE50M | 0.72 | 1670 | 3450 |
| WGE80BT* | — | 4800 | 3750 |
| WGE80M | 0.83 | 4900 | 3280 |
| SE70BT* | — | 2180 | 3650 |
| SE70M | 0.55 | 2775 | 3500 |
| CNE50M | 0.72 | 1800 | 3480 |
| Vegetable Peptone No. 1 | 0.72 | 2750 | 4280 |
| Hy-Soy | 0.60 | 2520 | 3300 |

*The peptone is insoluble.

Table 6 shows that NZ Soy, NZ-Soy BL4, and NZ-Soy BL7 were better than Vegetable Peptone No. 1. The best was NZ-Soy BL7, an insoluble peptone. All four were better than the rest.

TABLE VII
Comparison of the different non-animal peptones
that acted in earlier experiments as replacements
for Tryptone in TY fermentation medium.

| | | Toxin A | |
|---|---|---|---|
| | Cell growth | production (ng/ml) | |
| Peptone | (OD_{540 nm} at 24 hours) | 3 days | 5 days |
| Tryptone | 0.91 | 2709 | 7136 |
| Hy-Soy | 0.73 | 4608 | 5312 |
| NZ-Soy | 0.97 | 8832 | 11136 |
| NZ-Soy BL7* | — | 8704 | 12032 |
| SE50M | 0.88 | 2091 | 8128 |
| CNE50M | 0.72 | 3200 | 5216 |
| WGE80M | 0.77 | 7808 | 9472 |
| Plant Peptone E1 | 0.66 | 2645 | 3531 |
| Vegetable Peptone No. 1 | 0.75 | 1584 | 12544 |
| Soy Peptone Type II | 0.66 | 4960 | 5888 |
| Soy Peptone Type AC | 0.72 | 3477 | 6496 |
| Soy Peptone Type AB | 0.74 | 4384 | 5024 |
| Soy Peptone Type SL | 0.59 | 3627 | 4672 |
| NZ-Soy BL4 | 0.81 | 8032 | 9472 |

*The peptone is insoluble.

Table 7 shows that at 5 days Vegetable Peptone No. 1 and NZ-Soy BL7 were the best peptones for Toxin A production. NZ-Soy was almost as good and somewhat better than NZ-Soy BL4. At 3 days, NZ-Soy BL4 was best, and NZ-Soy and NZ-Soy BL7 were almost as good, but Vegetable Peptone No. 1 was poor. We have thus identified 4 good replacements for Tryptone. They are NZ-Soy BL7 (insoluble), NZ-Soy BIA, NZ-Soy, and Vegetable Peptone No. 1.

### Example 4

### Determine Whether Non-Animal Peptone Products can Replace Tryptone in Seed Medium for Cell Growth and Toxin A Production. (In Sections 4-9, the Working Cell Bank Stock Culture is Prepared in a Tryptone-Containing Medium)

TABLE VIII
Comparison of the following seed media: TYM medium
(control), TY medium, and VPY medium (Vegetable Peptone
No. 1 replacing Tryptone in TY medium)*.

| | Cell growth | | Toxin A | |
|---|---|---|---|---|
| | (OD_{540 nm} at 24 hours) | | production (ng/ml) | |
| Seed Media | 2^{nd} stage seed | Fermentation | 3 days | 5 days |
| TYM | 1.02 | 0.73 | 2080 | 4280 |
| TY | 1.02 | 0.93 | 500 | 1800 |
| VPY | 0.98 | 0.94 | 400 | 1100 |

*Fermentation was done in medium containing Vegetable Peptone No. 1 as a replacement for Tryptone in TY medium.

Table 8 shows that Toxin A production was lower when TY medium or TY containing Vegetable Peptone No. 1 as a Tryptone replacement was used as seed media. TYM was a much better seed medium for Toxin A production, despite the observation that growth was poorer in fermentation medium than with the other two seed media. TYM contains mannitol and glycerol. Thus, carbon sources in seed medium facilitate development of a good inoculum.

TABLE IX
Comparison of SYM seed medium (NZ-Soy BL4 as Tryptone
replacement in TYM seed medium) with TYM seed medium*.

| | Cell growth | | Toxin A | |
|---|---|---|---|---|
| | (OD_{540 nm} at 24 hours) | | production (ng/ml) | |
| Seed Media | 2^{nd} stage seed | Fermentation | 3 day | 5 day |
| TYM | 1.09 | 0.81 | 8032 | 9472 |
| SYM | 0.99 | 0.96 | 3712 | 5024 |

*Fermentation was done in NZ-Soy BL4 fermentation medium.

Table 9 shows that Toxin A production was lower in the NZ-Soy BL4 fermentation medium when NZ-Soy BL4 was used as a Tryptone replacement in the TYM seed medium. Although not as good as Tryptone, NZ-Soy BL4 led to about half the toxin production of the Tryptone seed medium.

TABLE X
Comparison of different non-animal peptones as
Tryptone replacements in TYM seed medium*.

| | Cell growth | | Toxin A | |
|---|---|---|---|---|
| | (OD_{540 nm} at 24 hours) | | production (ng/ml) | |
| Seed Media | 2^{nd} stage seed | Fermentation | 3 days | 5 days |
| Tryptone | 1.10 | 0.81 | 8320 | 10112 |
| Hy-Soy | 0.90 | 0.98 | 2192 | 2816 |
| NZ-Soy | 1.04 | 0.97 | 2720 | 3264 |
| NZ-Soy BL7** | — | 1.00 | 2080 | 2848 |
| SE50M | 0.97 | 0.99 | 2837 | 3072 |
| WGE80M | 0.89 | 0.99 | 2048 | 2816 |
| Plant Peptone E1 | 0.67 | 0.99 | 3221 | 3627 |
| Vegetable | 0.75 | 0.96 | 1964 | 3029 |
| Peptone No. 1 | | | | |
| Soy Peptone Type II | 0.87 | 0.95 | 2059 | 3552 |
| Soy Peptone | 1.00 | 0.97 | 3125 | 3755 |
| Type AC | | | | |
| Soy Peptone | 0.94 | 0.95 | 3605 | 3605 |
| Type AB | | | | |
| NZ-Soy BL4 | 0.95 | 0.99 | 3540 | 4181 |

*Fermentation was done in NZ-Soy BL4 fermentation medium.
**The peptone is insoluble.

Table 10 shows that the best non-animal peptone for seed medium was NZ-Soy BL-4. Plant Peptone E1, Soy Peptone Type AC, and Soy Peptone Type AB were next best for replacing the Tryptone in the TYM seed medium. However, Toxin A production was about 2.4 times higher when the seed medium contained Tryptone rather than NZ-Soy BL-4, both at 3 and 5 days.

TABLE XI
Comparison of different non-animal peptones as Tryptone
replacements in TYM seed medium (pH 7.5)*.

| | Cell growth | | Toxin A | |
|---|---|---|---|---|
| | (OD_{540 nm} at 24 hours) | | production (ng/ml) | |
| Seed Media | 2^{nd} stage seed | Fermentation | 3 days | 5 days |
| Tryptone | 1.11 | 0.79 | 6818 | 8416 |
| AMI Soy** | — | 0.99 | 2645 | 3541 |
| Hy-SoyT** | — | 0.97 | 2140 | 4235 |
| SE50MK | 0.96 | 1.01 | 2507 | 2976 |
| WGE80BT** | — | 0.96 | 2564 | 3211 |
| SE70BT** | — | 0.91 | 2720 | 4960 |
| SE70M** | — | 0.91 | 2837 | 4085 |
| CNE50M | 0.92 | 0.93 | 1984 | 2731 |
| Soy Peptone A1 | 0.95 | 0.93 | 2456 | 3712 |
| Soy Peptone A2 | 1.03 | 0.96 | 3285 | 4672 |
| Soy Peptone A3 | 1.04 | 0.94 | 3573 | 4704 |
| Plant Peptone | — | 0.94 | 2667 | 3529 |
| ET1** | | | | |
| Wheat Peptone E1 | 0.87 | 0.95 | 2816 | 3317 |
| NZ-Soy BL4** | — | 0.94 | 3467 | 3808 |

*Fermentation was done in NZ-Soy BL4 fermentation medium.
**The peptone is insoluble at ph 7.5.

Table 11 shows that the better non-animal peptones for seed media were Soy Peptone A3 (soluble), Soy Peptone A2 (soluble), SE70BT (insoluble), SE70M (insoluble), and HY-Soy T (insoluble) as replacements for the Tryptone in the TYM seed medium. However, Toxin A production was still much higher when the seed medium contained Tryptone (at both 3 and 5 days). The best non-animal peptone for seed medium considering both 3 and 5 days of fermentation was Soy Peptone A3, which yielded 52% of the Tryptone titer at 3 days and 56% at 5 days.

### Example 5

### Determining the Effect of Initial pH of Seed Medium on Cell Growth and Toxin A Production

TABLE XII
Comparison of initial pH of NZ-Soy BL4 seed medium for cell
growth and Toxin A production in NZ-Soy BL4 fermentation medium.

| | | Cell growth | | Toxin A | |
|---|---|---|---|---|---|
| Test | pH of | (OD₅₄₀ nm at 24 hours) | | production (ng/ml) | |
| No. | seed medium | 2^{nd} stage seed | Fermentation | 3 days | 5 days |
| I | 6.8 | 0.95 | 0.99 | 3540 | 4181 |
| | 7.5 | —* | 0.93 | 5152 | 5120 |
| | 8.5 | —* | 0.95 | 3349 | 4395 |
| II | 6.8 | 0.98 | 0.92 | 2160 | 3050 |
| | 7.5 | —* | 0.94 | 3488 | 3808 |

*The peptone was insoluble at these higher pH values.

Table 12 shows that cell growth was similar in the fermentation medium, but Toxin A production increased when the pH of the seed medium was raised from 6.8 to 7.5. Toxin A production decreased when the pH of the seed medium was further increased to 8.5. From this experiment on, the initial pH of the seed media was 7.5 for control.

TABLE XIII
Comparison of initial pH of Soy Peptone A3 seed medium for cell
growth and Toxin A production in NZ-Soy BL4 fermentation medium.

| | Cell growth | | Toxin A | |
|---|---|---|---|---|
| | (OD_{540 nm} at 24 hours) | | production (ng/ml) | |
| pH of seed medium | 2^{nd} stage seed | Fermentation | 3 days | 5 days |
| 7.5 | 1.10 | 0.77 | 8480 | 10752 |
| 8.5 | 1.10 | 0.73 | 9856 | 11904 |

Table 13 shows a slightly positive effect on Toxin A production at both 3 and 5 days when pH of Soy Peptone A3 seed medium was increased from 7.5 to 8.5.

### Example 6

### Determining the Effect of Different Concentrations of Nitrogen Source in Seed Media on Cell Growth and Toxin A Production

TABLE XIV
Comparison of different concentratioin of NZ-Soy
BL4 in seed medium for cell growth and Toxin A
production in NZ-Soy BL4 fermentation medium.

| | Cell growth | | Toxin A | |
|---|---|---|---|---|
| NZ-Soy BL4 in | (OD_{540 nm} at 24 hours) | | production (ng/ml) | |
| seed medium (g/L) | 2^{nd} stage seed | Fermentation | 3 days | 5 days |
| 24 (control) | 0.95 | 0.99 | 3540 | 4181 |
| 48 | 0.95 | 0.93 | 3904 | 3755 |
| 96 | 1.00 | 0.94 | 5851 | 4704 |

Table 14 shows that cell growth and Toxin A production were not markedly affected when NZ-Soy BL4 in seed medium was increased from 24 g/L up to 96 g/L.

### Example 7

### Determining the Effect of Inoculum Concentration Used for Fermentation Stage on Cell Growth and Toxin A Production

TABLE XV
Comparison of inoculum concentration in fermentation
stage on cell growth and Toxin A production
in NZ-Soy BL4 fermentation medium.

| | | Toxin A | |
|---|---|---|---|
| Inoculum | Cell growth | production (ng/ml) | |
| concentration (%) | (OD_{540 nm} at 24 hours) | 3 days | 5 days |
| 0.5 (control) | 0.76 | 12288 | 11008 |
| 1.0 | 0.75 | 8576 | 11904 |
| 5.0 | 0.77 | 8896 | 12928 |

Table 15 shows a negative effect on Toxin A production at 3 days when increased inoculum volume was used for fermentation. At 5 days, there was a slight stimulation. It would appear that 0.5% is satisfactory as an inoculum concentration for fermentation.

### Example 8

### Determining the Effect of Reduced Iron Powder and Soluble Iron Compounds Added to Second Stage Seed Media on Cell Growth and Toxin A Production

TABLE XVI
Effect of reduced iron powder (0.5 g/L) in the second
stage seed medium on cell growth and Toxin A production
in NZ-Soy BL4 fermentation medium.

| | Cell growth | | Toxin A | |
|---|---|---|---|---|
| | (OD_{540 nm} at 24 hours) | | production (ng/ml) | |
| Seed media | 2^{nd} stage seed | Fermentation | 3 days | 5 days |

Test I

| | | | | |
|---|---|---|---|---|
| Soy Peptone A3 | 1.03 | 0.76 | 12288 | 11008 |
| Soy Peptone A3 + | —* | 0.72 | 11264 | 15744 |
| iron pdr. | | | | |

Test II

| | | | | |
|---|---|---|---|---|
| Soy Peptone A3 | 1.10 | 0.77 | 8480 | 10752 |
| Soy Peptone A3 + | —* | 0.71 | 11008 | 14208 |
| iron pdr. | | | | |
| NZ-Soy BL4 | —** | 0.90 | 2548 | 3703 |
| NTZ-Soy BL4 + | —* | 0.87 | 3776 | 5568 |
| iron pdr. | | | | |

*The iron powder is insoluble.
**The peptone was insoluble at pH 7.5.

Table 16 shows that Toxin A production was markedly increased at 5 days when 0.5 g/L reduced iron powder was added into the second stage seed medium. In all cases but one, it also increased production at 3 days.

TABLE XVII
Comparison of different iron compounds in second stage
Soy Peptone A3 seed medium on cell growth and Toxin
A production in NZ-Soy BL4 fermentation medium.

| Iron compounds | Cell growth | | Toxin A | |
|---|---|---|---|---|
| in second stage | (OD_{540 nm} at 24 hours) | | production (ng/ml) | |
| seed medium (g/L) | 2^{nd} stage seed | Fermentation | 3 days | 5 days |
| None | 1.10 | 0.77 | 8480 | 10752 |
| Reduced iron | — | 0.71 | 11008 | 14208 |
| powder (0.5)* | | | | |
| FeSO₄•7H₂O (0.04) | 3.05 | 0.75 | 8704 | 12160 |
| Ferrous | 1.02 | 0.75 | 9728 | 13184 |
| gluconate (0.2) | | | | |

*The iron powder is insoluble.

Table 17 confirms that Toxin A production is increased both at 3 and 5 days when 0.5 g/L reduced iron powder is added into the second stage seed medium. Toxin A production was increased at 5 days when 40 mg/L FeSO₄ and 200 mg/L ferrous gluconate were added into the second stage seed medium, but FeSO₄ was not stimulatory at 3 days.

Iron powder is thus a useful additive to seed media containing vegetable peptones. If the insolubility is a problem, ferrous gluconate can be used.

### Example 9

### Determining the Effect of Vitamin B₁₂ Added to Second Stage Seed Medium on Cell Growth and Toxin A Production

TABLE XVIII
Effect of adding vitamin B₁₂ in Soy Peptone A3 second
stage seed medium on cell growth and Toxin A production
in NZ-Soy BL4 fermentation medium.

| | Cell growth | | Toxin A | |
|---|---|---|---|---|
| Vitamin B₁₂ | (OD_{540 nm} at 24 hours) | | production (ng/ml) | |
| (μg/L) | 2^{nd} stage seed | Fermentation | 3 days | 5 days |

seed medium without mannitol and glycerol

| | | | | |
|---|---|---|---|---|
| 0 | 0.86 | 0.93 | 2667 | 3168 |
| 0.5 | 0.92 | 0.92 | 3349 | 3913 |
| 5 | 0.92 | 0.91 | 3733 | 3861 |
| 50 | 0.97 | 0.91 | 3275 | 3989 |

Seed medium with mannitol and glycerol

| | | | | |
|---|---|---|---|---|
| 0 | 1.03 | 0.76 | 12288 | 11008 |
| 0.5 | 1.08 | 0.76 | 7904 | 11392 |
| 5 | 1.10 | 0.80 | 7968 | 9632 |
| 50 | 1.08 | 0.78 | 7616 | 11008 |

Table 18 shows that Toxin A production slightly increased when Vitamin B₁₂ was added into the Soy peptone A3 second stage seed medium that did not contain mannitol and glycerol. However, when seed medium contained mannitol and glycerol, Vitamin B₁₂ had a negative effect on Toxin A production at 3 days but not at 5 days.

### Example 10

### Determining Whether a Non-Animal Peptone can Replace Tryptone in Medium for Preparation of Working Cell Bank (WCB) Stock Cultures of C. difficile VPI 10463

TABLE XIX
Comparison of vegetable peptones in media for preparation
of stock cultures and seed media on cell growth and Toxin
A production in NZ-Soy BL4 fermentation medium.

| | | Cell growth | | Toxin A | |
|---|---|---|---|---|---|
| | | (OD_{540 nm} at 24 hours) | | production | |
| | Seed | 2^{nd} | Fermen- | (ng/ml) | |
| WCB peptone | media | stage seed | tation | 3 days | 5 days |

Test I

| | | | | | |
|---|---|---|---|---|---|
| Tryptone | TYM | 1.10 | 0.86 | 4576 | 5568 |
| Soy Peptone A3 | SYM** | 0.86 | 0.93 | 12288 | 11008 |

Test II

| | | | | | |
|---|---|---|---|---|---|
| Tryptone | TYM | 1.13 | 0.73 | 5408 | 6499 |
| Soy Peptone A3 | SYM | 1.10 | 0.77 | 8480 | 10752 |
| NZ-Soy BL4* | ZYM*** | — | 0.90 | 2548 | 3703 |

*The peptone is insoluble at pH 7.5.
**Soy Peptone A3 replaced Tryptone in TYM.
***NZ-Soy BL4 replaced Tryptone in TYM.

Table 19 shows that the medium for preparing working cell bank stock culture WCB8.9.0-SPA3 containing Soy Peptone A3 was excellent. Using it, Toxin A production was much higher with Soy Peptone A3 as the Tryptone replacement in TYM seed medium for first stage seed culture and second stage seed culture than the control situation using the old cell bank stock culture prepared with Tryptone in TYM. Toxin A production was much lower with NZ-Soy BL4 than with Tryptone or Soy Peptone A3 in working cell bank stock culture medium.

The results show that vegetable peptones can be used in all 4 stages of the process, i.e., working cell stock culture preparation medium, first stage seed medium, second stage seed medium, and fermentation medium.

### Example 10

### The Effect of Raising the Initial pH of Fermentation Medium on Growth and Toxin A Production

TABLE XX
Effect of pH of fermentation media on cell growth and
Toxin A production with Soy Peptone A3 seed medium.

| | | | Toxin A | |
|---|---|---|---|---|
| Fermentation | Initial | Cell growth | production (ng/ml) | |
| Media | pH | (OD₅₄₀ nm at 24 hours) | 3 days | 5 days |
| Soy Peptone A3 | 6.8 | 0.69 | 1024 | 5504 |
| Soy Peptone A3 | 7.5 | 0.69 | 5024 | 6240 |
| NZ-Soy BL4 | 6.8 | 0.77 | 8480 | 10752 |
| NZ-Stoy BL4 | 7.5 | —* | 6656 | 9056 |

*The peptone is insoluble at pH 7.5.

Table 20 shows that increasing pH of NZ-Soy BL4 fermentation medium from 6.8 to 7.5 decreased Toxin A production. Increasing the pH of Soy Peptone A3 fermentation medium from 6.8 to 7.5 increased Toxin A production. Since NZ-Soy BLA is a better Tryptone replacement in fermentation medium than Soy Peptone A3, NZ-Soy BL4 fermentation medium should be used in the future and the pH should not be increased to 7.5.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method of culturing Clostridium difficile, said method comprising growing said Clostridium difficile in a medium that is substantially free of animal-derived products.

2. The method of claim 1, wherein said medium comprises a compound derived from a vegetable.

3. The method of claim 2, wherein said vegetable is a soybean.

4. The method of claim 3, wherein said compound is hydrolyzed soy.

5. The method of claim 1, wherein said medium further comprises an iron source.

6. The method of claim 1, wherein said culturing is carried out under anaerobic conditions.

7. The method of claim 1, wherein said Clostridium difficile is being grown as a seed culture.

8. The method of claim 7, wherein said seed culture started by inoculation from a stock culture that was grown in medium that was substantially free of animal-derived products.

9. The method of claim 1, wherein said Clostridium difficile is being grown as a fermentation culture.

10. The method of claim 9, wherein said fermentation culture was inoculated from a seed culture that was grown in medium that was substantially free of animal-derived products.

11. The method of claim 10, wherein said seed culture was a second seed culture.

12. The method of claim 9, further comprising isolating Clostridium difficile Toxins from said medium.

13. A method of obtaining Clostridium difficile toxins, said method comprising the steps of: culturing Clostridium difficile in a first medium under conditions that facilitate growth of Clostridium difficile, wherein said first medium is substantially free of animal-derived products; inoculating a second medium with all or a portion of said first medium after said culturing, wherein said second medium is substantially free of animal-derived products; culturing said inoculated second medium under conditions that facilitate growth of Clostridium difficile and toxin production; and isolating Clostridium difficile toxins from said second medium.

14. The method of claim 13, wherein said first and second media comprise a compound derived from a vegetable.

15. The method of claim 14, wherein said vegetable is a soybean.

16. The method of claim 15, wherein said compound is hydrolyzed soy.

17. The method of claim 13, wherein said culturing of said first or second media comprising Clostridium difficile is carried out under anaerobic conditions.

18. The method of claim 13, wherein the culturing in said first medium was started by inoculation of said first medium with a previous Clostridium difficile culture that was cultured in medium that was substantially free of animal-derived products.

19. The method of claim 18, wherein said previous culture was a stock culture.

20. The method of claim 18, wherein said previous culture was a previous seed culture that was obtained by inoculation from a stock culture that was prepared by culture in medium that was substantially free of animal-derived products.
